(19) United States
(12) Patent Application Publication
(10) Pub. No.: US 2014/0100797 A1
(43) Pub. Date: Apr. 10, 2014

(54) SYSTEM AND METHOD FOR DETERMINING THE PRODUCTION OF WELLS

(71) Applicants: Mihira Narayan Acharya, Ahmadi (KW); Mashari Mohammed Abdul Rahman Al-Awadi, Ahmadi (KW); Ahmad Jaber Al-Eidan, Ahmadi (KW)

(72) Inventors: Mihira Narayan Acharya, Ahmadi (KW); Mashari Mohammed Abdul Rahman Al-Awadi, Ahmadi (KW); Ahmad Jaber Al-Eidan, Ahmadi (KW)

(21) Appl. No.: 13/647,191

(22) Filed: Oct. 8, 2012

(51) Int. Cl. G01N 33/00

(52) U.S. Cl. CPC G01N 33/0031
USPC 702/24

(57) ABSTRACT

According to one embodiment, a system for determining hydrocarbon production may be disclosed. The system may include one or more processors and a memory comprising logic. The memory comprising logic may be operable to determine an accumulated unit density of a reservoir unit from a density log. The memory comprising logic may be further operable to determine a thickness of the reservoir unit and determine a density footage of the reservoir unit based on the accumulated density and the thickness. The memory comprising logic may also be operable to generate a total organic carbon content per density footage based on the density footage. In accordance with certain embodiments, the memory comprising logic may be operable to determine a potential production of hydrocarbons based on the total organic carbon content per density footage of the reservoir unit.

### BACKGROUND OF THE INVENTION

1. Field of the Invention

The invention relates generally to determining the production of wells.

2. Description of Related Art

Determining the oil and gas production success of wells is a challenge, particularly in unconventional resource plays. The oil and gas reservoirs buried deep in the rock formations of unconventional resource plays, such as those resource plays with lower organic rich laminated reservoir units, may contain vast amounts of hydrocarbon resources, but the well sites selected using traditional production indicators often yield poor results. Oil exploration and production companies often fail to produce liquid hydrocarbons at most predicted well sites when relying on such traditional indicators, and often spend millions of dollars on those unproductive well sites.

Traditional petrophysical interpretation methods and integrated formation evaluation methods may not sufficiently explain the behavior of these unconventional resource plays. In conventional resource plays, porous rock units with high fluid saturation and high fracture rates usually indicate likely hydrocarbon production. However, no positive correlation may be drawn between this formation composition and the production success of many unconventional resource play wells.

Accurately determining the production success of well drill sites is critical to achieving cost-effective drilling operations in unconventional resource plays. Accurately determining production success ensures the sunk costs of unproductive well sites do not compromise the profitability of drilling in the region and enables oil and gas production companies to efficiently schedule resources.

### SUMMARY OF THE INVENTION

According to one embodiment, a system for determining hydrocarbon production may be disclosed. The system may include one or more processors and may include a memory comprising logic. The memory comprising logic may be operable to determine an accumulated unit density of a reservoir unit from a density log. The memory comprising logic may be further operable to determine a thickness of the reservoir unit and determine a density footage of the reservoir unit based on the accumulated density and the thickness. The memory comprising logic may also be operable to generate a total organic carbon content per density footage based on the density footage. In accordance with certain embodiments, the memory comprising logic may be operable to determine a potential production of hydrocarbons based on the total organic carbon content per density footage of the reservoir unit.

Technical advantages of the disclosure may include one or more of facilitating efficient well site planning without requiring new data collection, enabling accurate well categorization and production planning in traditionally unpredictable production regions, and enabling classification of unconventional petroleum stores.

### BRIEF DESCRIPTION OF THE DRAWINGS

FIG. 1 illustrates a block diagram of a system for determining the production success of wells.

FIG. 2A illustrates bulk density logs in the system for determining the production success of wells of FIG. 1.

FIG. 2B illustrates the bulk density logs for wells that produced hydrocarbons in the system for determining the production success of wells of FIG. 1.

FIG. 2C illustrates the bulk density logs for wells that did not produce hydrocarbons in the system for determining the production success of wells of FIG. 1.

FIG. 3 illustrates a bulk density log, a derived total organic carbon content table and a core sample measured total organic carbon content table.

FIG. 4 illustrates the steps in the accumulate function in the system for determining the production success of wells of FIG. 1.

FIG. 5 illustrates the transform function for calculating the total organic carbon content from density footage in the system for determining the production success of wells of FIG. 1.

FIG. 6 illustrates a productivity index formula for calculating the expected productivity of a well.

FIG. 7 illustrates a plot of productivity indexes against total organic carbon content of productive wells.

FIG. 8 illustrates a histogram plot of an average total organic carbon content of the wells in the system for determining the production success of wells of FIG. 1.

FIG. 9A illustrates a total organic carbon content value per density footage contour map representing the total organic carbon content per density footage of predicted wells in the system for determining the production success of wells of FIG. 1.

FIG. 9B illustrates a total organic carbon content value per density footage contour map representing the total organic carbon content per density footage of the productive wells in the system for determining the production success of wells of FIG. 1.

### DETAILED DESCRIPTION

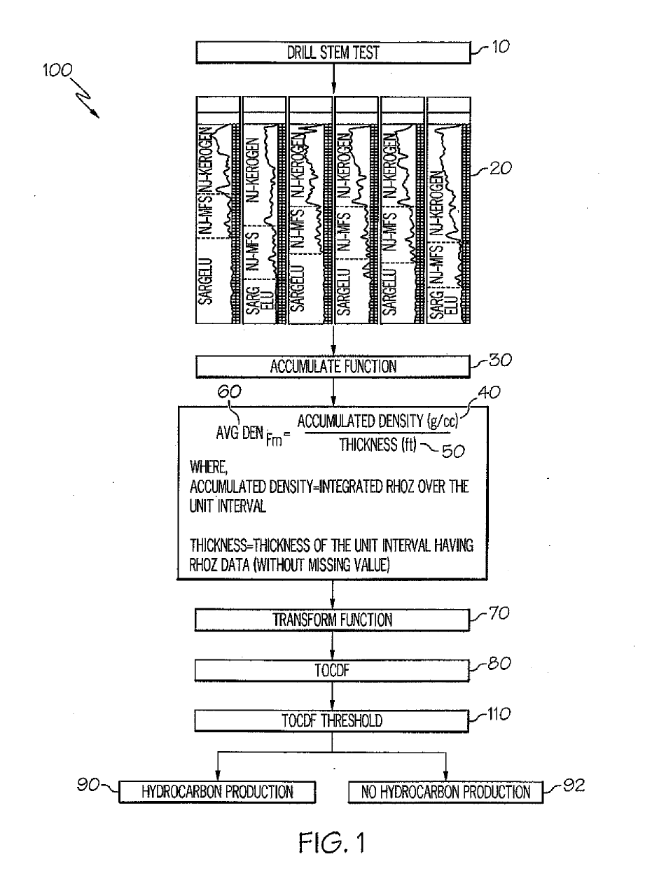

Embodiments of the present disclosure and their features and advantages may be understood by referring to FIGS. 1-9; like numerals being used for corresponding parts in the various drawings.

FIG. 1 illustrates a block diagram of a system 100 for determining the production success of wells that may be located in, for example, unconventional resource plays.

As used herein, production success refers to whether a well drilled in a specified geographic site may be expected to produce hydrocarbons, e.g., gas or condensate liquid oil, to an extent that justifies a commercial investment in the well. The productivity index may refer to a ratio of an average liquid hydrocarbon flow rate with respect to the pressure draw-down of a well.

In system 100, a drill stem test 10 (DST) may be conducted at a geographic region in a resource play. The results of DST 10 may be forecasted from an acquired bulk density log 20 associated with the geographic region. Thus, bulk density log 20 may comprise data from a reservoir unit. An accumulate function 30 may accumulate density values from bulk density log 20 along the reservoir unit and may produce an accumulated density 40 in grams per cubic centimeters. A unit thickness 50 may comprise an average thickness in feet along the reservoir. Unit thickness 50 may be determined from bulk density log 20. A density footage 60 may comprise a quotient of accumulated density 40 divided by unit thickness 50. A transform 70 may be applied to density footage 60 to compute a total organic carbon content per density footage (TOCDF) 80. A production success 90 may be determined for the geographic region using TOCDF 80. For example, production success may be predicted if TOCDF 80 is less than or equal to a TOCDF threshold 110. A production failure 92 may be determined for the geographic region if TOCDF 80 is greater than TOCDF threshold 110.

TOCDF 80 is an organic richness measure that may be derived in a two step process. An average density per foot thickness, or density footage, of the reservoir unit may be calculated by accumulating weighted bulk density values from bulk density log 20 across the reservoir unit and dividing by unit thickness 50 as measured in feet. Bulk density log 20 may comprise information about lithographic units in the region. For example, inspection of the density log 20 may reveal the presence or location of a reservoir unit. Density footage 60 may be input into a transform equation to obtain TOCDF 80. If TOCDF 80 for a particular well site is less than or equal to TOCDF threshold 110, the well site may be expected to produce hydrocarbons. TOCDF threshold 110 may comprise a unitized weight % value of total organic carbon content calibrated for a specific region. In accordance with a particular embodiment of the present disclosure, TOCDF threshold 110 may comprise a value of 2.45 weight %.

In certain embodiments, the disclosure may be applicable to unconventional resource plays. Unconventional resource plays may comprise, for example, a kerogen unit and reservoir unit. The kerogen unit may comprise an organic rich laminated kerogen layer. The kerogen unit may comprise argillaceous and calcareous clay, and may contain high uranium content as classified by spectral gamma ray subsurface scanning logs.

The reservoir unit may comprise a porous and fractured lower organic rich laminated reservoir layer. The reservoir unit may comprise high organic richness as derived from a bulk density log. Traditional production indicators may not accurately determine the production success of this reservoir unit. The reservoir unit may be disposed beneath the kerogen unit.

The embodiments in this disclosure may describe resource plays comprising a lower organic rich laminated reservoir unit, but the disclosure may be applied to resource plays where traditional formation evaluation procedures fail to accurately determine well production success of DST 10.

Traditional reservoir production indicators may comprise one or more of high porosity, high fluid saturation, and high fracture rates. Traditional reservoir production indicators may not accurately determine whether a resource play may produce hydrocarbons. In conventional resource plays, a region comprising a rock unit having one or more of the production indicators (e.g., high porosity, high fluid saturation, and high fracture rates) indicates the resource play may produce hydrocarbons. Traditional reservoir production indicators may be determined to be present by analyzing bulk density logs of the resource play.

DST 10 may be conducted in a well for a particular zone of interest or reservoir rock after drilling and logging lithographic information, including bulk density log 20, to collect information about the reservoir's hydrocarbon productivity. A drill string may drill a bore hole in the ground. In certain embodiments, the drill string may comprise one or more of a drill, a logging while drilling tool (LWD) and a measurement while drilling (MWD) tool. The drill may comprise a drill bit and may drill through the earth, which may create a bore hole. The MWD tool may scan lithographic layers of a region prior to DST 10. The LWD tool may produce bulk density log 20, and may store bulk density log 20 in a memory on the drill string. The LWD tool may relay bulk density log 20 to a receiver on the surface near the bore hole. Bulk density log 20 may comprise information about the density of minerals and fluids enclosed in pore spaces along the length of a rock formation adjacent to a borehole. In other embodiments, bulk density log 20 may be collected from a conventional logging tool, e.g., wireline or pipe conveyed, as well as from a logging while drilling device. A drilling service provider may provide bulk density log 20 to an oil production company from drilled wells that may survey rock formations across a region. In conventional resource plays, the oil production company may select the appropriate geographic location for drilling based on bulk density log 20.

Bulk density log 20 may comprise standard resolution formation density logs (RHOZ). The gamma ray log may indicate natural radioactivity of the rock formation. When the gamma ray line deflects to the left, the rock unit may be a reservoir quality rock unit. When the gamma ray line deflects to the right, the rock unit may be a shale unit, traditionally unsuitable for hydrocarbon extraction. But high organic rich shale or kerogen rock with high gamma ray values may be a prolific source rock for hydrocarbons. System 100 may calculate TOCDF 80 of the reservoir unit based on bulk density log 20, and may accurately determine the production success of the region.

System 100 may determine the production success of the region without conducting additional DSTs to collect information. Bulk density logs may be available for regions where hydrocarbons have been detected. System 100 may use these readily available logs in determining the production success of wells.

Transform function 70 may provide an accurate estimation of TOCDF 80. TOCDF 80 may be measured in a lab by heating source rock samples and measuring the amount of carbon dioxide released. This process may require time and laboratory expenses. Transform function 70 may provide an accurate estimate of TOCDF 80. Transform function 70 may require information comprising bulk density log 20 but may not require laboratory experiments or equipment.

Resource plays may be difficult for project planning and scheduling because of the unpredictable nature of production in those regions. Drilling equipment may be moved to new sites, and significant planning may be required to begin drilling operations at a new site. Government compliance and service contracts may add to the complexity. Establishing drilling operations at an unproductive well site may cost oil production companies millions of dollars. System 100 may save exploration time and may eliminate the cost of drilling unproductive well locations. As a result, resource plays may be accurately categorized based on expected production success and may be scheduled for efficient hydrocarbon extraction.

In a particular embodiment, characterizing unconventional reservoirs may be a challenge. Specific reservoir fields may have been tested and may have been found to be prolific source-rocks and producers of gas, condensate, and volatile oil. DST results at some wells may be successful, while other wells may be unsuccessful despite repeated stimulation attempts. The resulting success rate may be 50%. The region may thus be categorized as a geologically-complex, naturally-fractured, tight gas and condensate reserve.

For example, the kerogen unit in the region may be composed of highly organic rich argillaceous and calcerous clay, and may be represented by very high total gamma ray values associated with high uranium on spectral gamma logs. The limestone unit, underlying the kerogen and overlaying the shale unit, may generally be tight and occasionally fractured. Conventional characterization by multi-disciplinary data integration and models may not explain the test results. Thus, the key factor in determining which reservoirs will produce may lie outside the scanned parameters, such as structural position, fractures and formation damage. Conventional petrophysical interpretation and integrated formation evaluation methods may fail to explain the unique behavior of such reservoirs.

The TOCDF for the kerogen and limestone units in the above example may characterize the relationship between the successful and unsuccessful DSTs. Thus, the key to successful hydrocarbon production in such regions may be based on the organic richness of the regions, which may be derived from wireline density logs. This approach may have successfully predicted several completed wells and may aid in navigating 3 dimensional model building workflow steps for seismic reservoir descriptions. The approach may further shape future development strategies in unconventional reservoir regions.

In another embodiment, conventional drill stem well test results in sub-salt formations in gas fields spread across 1700 sq km, may have displayed a rate of flow to no-flow of 1:1 in 14 vertical exploratory wells out of the 29 wells drilled. The controlling factor, or principal reservoir parameter component, for such well test trends in the complex reservoirs, composed of sandwiched organic rich kerogen (unconventional and prolific source rock) unit with tight fractured carbonate sequences, may have been unknown. The conventional approaches and reservoir parameters, such as porosity, saturations, fractures etc., may have fallen short of determining the outcome of conventional test results, adding more uncertainty in the reservoir characterization. A study may have been undertaken to find a quantifiable reservoir parameter as a principal controlling component calibrated with an observed DST trend and that may be used as a predictive tool for well test decisions in future wells and may help define a befitting development strategy accordingly.

A parameter TOCDF may be the average total organic carbon content (TOC) derived using average density footage as a replacement of the average formation density parameter in transform function 70. A unitized quantified organic richness parameter, TOCDF, using density footage in transform function 70 may be used instead of simple average formation density, where, density footage for the geological sub-unit may be calculated using a tp-accumulate module of Geolog 7.0 from bulk density log 20. Accumulated density 40 in grams per cubic centimeters (g/cc) for the sub-unit may be divided by unit thickness 50 in feet to derive density footage 60 of the corresponding sub-unit, which may in turn be used for computing TOCDF 80.

The tp-accumulate module may be described below:

1: tp-accumulate: Geolog
tp_accumulate module accumulates an input log by integration.
It sums or accumulates an input log across intervals.
The sum is reset to zero at each new interval.
The output accumulation is written to both the Interval set and the Wire set.
The accumulated log output to the Wire set will always have missing values if the input log is missing, but
The accumulated log written to the Interval set will always have the cumulative total over the interval.
Accumulations may be performed either forward or reversed in depth.
Accumulation is defined as:

accum[i]=accum[i−1]+log_in[i]*thickness
Where,
thickness=half_thickness_above+half_thickness_below
and
half_thickness_above=[depth[i]−depth[i−1]]/2
and
half_thickness_below=[depth[i+1]−depth[i]]/2

The geological and stratigraphic units may have been correlated in all 14 wells under the study in the example, and may establish the reservoir unit's thickness consistency, which may be divided in two sub units. The top sub-unit may be highly organic rich with all 14 wells comprising an average TOCDF between 4.2 weight % and 10.2 weight %, and an average thickness of 50 ft. The bottom sub-unit may be lower organic rich laminated (LORL), with average TOCDF between 0.03 weight % and 4 weight %, and average thickness of 30 ft. A reasonable match of TOCDF with the average TOC from available core data may have been used for calibration. The TOCDF of the top sub-unit may show a strong trend fit with the DST results where positive and successful results may have been displayed in wells having top sub-unit TOCDF less than 2.45 weight %.

The discriminating principal controlling parameter, TOCDF, may have been determined for the top unit in the 14 tested wells and the window for successful DST results may now be applied as a predicting tool for future wells and may further be applied for other wells which were not tested in the fields. This workflow and TOCDF parameter may have been successfully tested and applied in both predicting positive DST outcomes in the study fields and in verifying negative test predictions in a work-over rig intervention in November 2009, consisting of 63 days of operation and costing $4.3 million. This workflow and approach may be applied elsewhere in similar reservoirs.

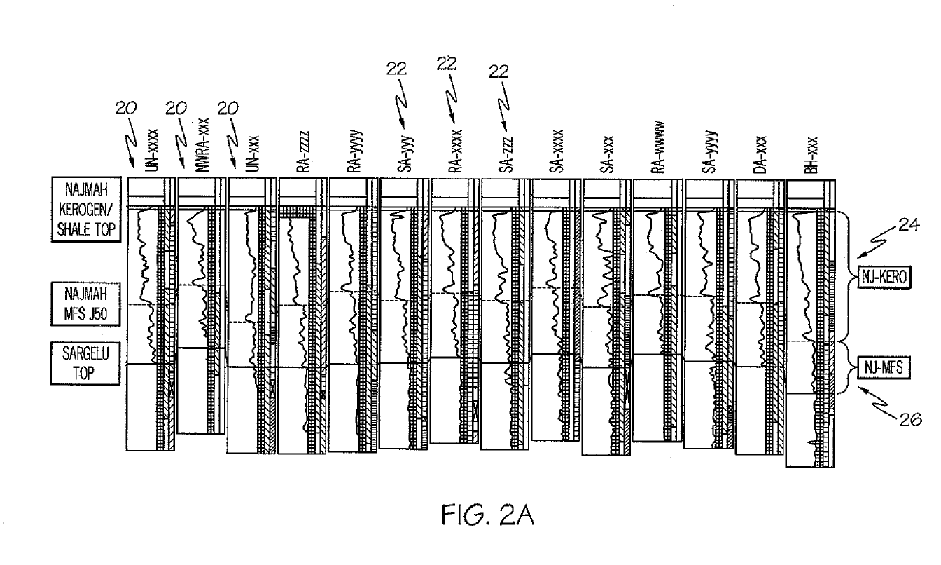

FIGS. 2A-C illustrate bulk density logs in the system 100 of FIG. 1. Bulk density log 20 may comprise data from a geographic region 22 in a resource play. The resource play may comprise a kerogen unit 24. Kerogen unit 24 may physically comprise highly organic rich argillaceous and calcareous clay. The resource play may comprise a reservoir unit 26. Reservoir unit 26 may comprise a lower organic rich laminated layer. Reservoir unit 26 may comprise highly fractured limestone. Density log 20 may display traditional production success indicators that may lead to mis-categorization of the region when using traditional petrophysical evaluation methods. In conventional resource plays, traditional production indicators may enable determining whether a well will produce hydrocarbons by analyzing bulk density log 20. However, in some resource plays, traditional production success indicators may be ineffective.

For example, the graphs in FIG. 2B illustrate bulk density logs 220, like bulk density log 20 in system 100, for test wells in resource plays that have successfully produced hydrocarbons. The graphs in FIG. 2C illustrate bulk density logs 320 for test wells in resource plays that did not successfully produce hydrocarbons. Traditional factors indicated that all the well sites corresponding to the bulk density logs in FIGS. 2B-C would successfully produce hydrocarbons. FIG. 2B illustrates that traditional production success indicators may not reliably determine the production success of wells.

In reference to FIG. 2B, density log 220 may comprise 3 primary lithographic units that may be used in a traditional production success analysis. Unit 230 may indicate a low density formation with low interconnected and effective porosity. Such formations may indicate a kerogen unit. Bulk density log 220 may indicate dense formations when the RHOZ value shifts to the right. Bulk density log 220 may indicate a low porosity region where the RHOZ value shifts to the right. Unit 240 may indicate a low organic rich laminated limestone and kerogen unit. Such formations may indicate a reservoir unit. Reservoir units may be optimal for hydrocarbon extraction because hydrocarbon from kerogen layers above may reside in the pores or fractures present in a reservoir unit. Bulk density log 220 may indicate porous formations where the neutron porosity value shifts to the left. Unit 250 may indicate a high density tight limestone unit with low porosity. The logs in FIG. 2C may indicate the same regions.

The logs displayed in FIGS. 2A-C are an example of a typical series of DST plots that a producer may receive from a service provider. The density logs in FIGS. 2A-C may be used in the examples and proofs that follow, but the disclosure should not be construed as limited to these regions.

A drilling service provider may provide bulk density log 20 to a production company from previous regional surveys conducted from existing offset wells. The production company may survey regions to obtain bulk density logs 20. One or more of LWD and MWD tools may survey resource plays and produce bulk density logs 20.

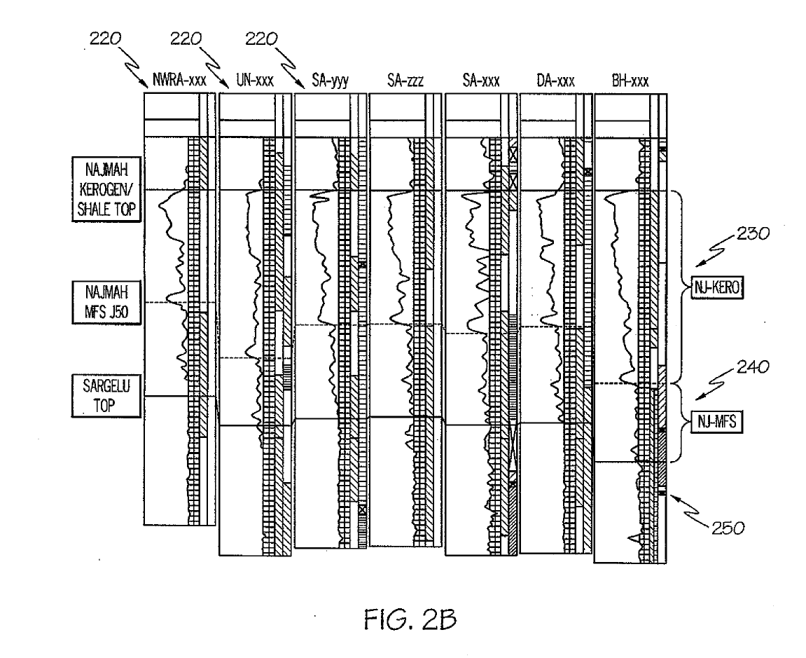

FIG. 3 illustrates a density log 400, a density log derived TOCDF table 500, and a core sample TOCDF table 600 calculated from core plug samples for each equivalent unit. FIG. 3 may demonstrate the accuracy of deriving the TOCDF for a reservoir unit from bulk density log 400 as compared to an average of total organic carbon content measurements from core samples. A log derived TOCDF content value 510 of the upper kerogen unit may be validated against the average total organic carbon content measurement of core plug sample 610 from the equivalent kerogen unit. The tables may be split into two units, a kerogen unit 520 and a reservoir unit 530, and the density log derived TOCDF content value may be computed for each unit in each region. The derived TOCDF values in table 500 may be derived from density log 400. Core plug samples corresponding to each entry in table 600 may be collected for each region. The values between table 500 and table 600 may correspond within an acceptable error range. This may validate the use of density logs in deriving the TOCDF content of units as an acceptable substitute for actual core samples.

Core sample measurements may be subject to a chemical analysis to determine their TOCDF. This process may require heating the core sample rock in a furnace to release carbon dioxide, then measuring the carbon dioxide with a thermal conductivity detector or infrared spectroscopy. This process may require time and require laboratory analysis. The log derived TOCDF may provide an accurate estimation of TOCDF without requiring chemical analysis or laboratory experiments.

The accuracy of the derived TOCDF content may eliminate the need for actual core samples to be drawn at each prospective well site. While slightly more accurate determinations of TOCDF content may be obtained by actual core samples, the reduced cost of deriving TOCDF content may render obtaining actual core samples inefficient.

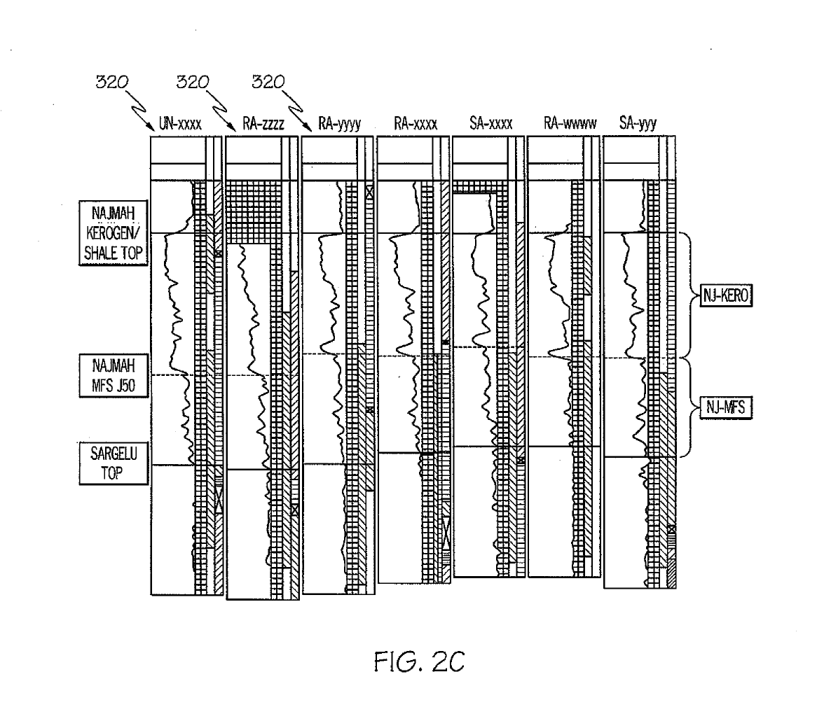

FIG. 4 illustrates the steps in accumulate function 30 in the system for determining the production success of wells of FIG. 1. Accumulate function 30 may calculate the accumulated density of the bulk density logs for each geographic region in the density footage calculation. Accumulate function 30 may sum the input density log across each interval. The function may reset the sum calculation at each new unit interval. The output accumulation may be written to both the interval set and the wire set. The accumulated log output to the wire set may contain the same missing values that the input log is missing, but the interval set accumulation log may always have the cumulative density total over the interval.

Accumulate function 30 may comprise an accumulation array 700. Accumulation array 700 may contain indices for each interval that a measurement is available for the reservoir region from the bulk density log. Accumulate function 30 may store a value at an index in accumulation array 700. The value stored may comprise a bulk density value 705 at the index multiplied by a thickness value 710. Bulk density value 705 may comprise the density value of a rock formation at an interval. The interval may correspond to the index. Thickness value 710 may comprise the average thickness of the unit at the interval. Thickness value 710 may comprise a half thickness above 715 and a half thickness below 720. Half thickness above 715 may comprise the average thickness of the interval over the area between the interval and the previous interval. Half thickness below 720 may comprise the average thickness of the interval over the area between the interval and next interval. Accumulate function 30 may be equivalent to a definite integral of the bulk density log multiplied by the average unit thickness, calculated over the unit. Accumulate function 30 may be calculated with a software script or a petrophysical geological analysis tool.

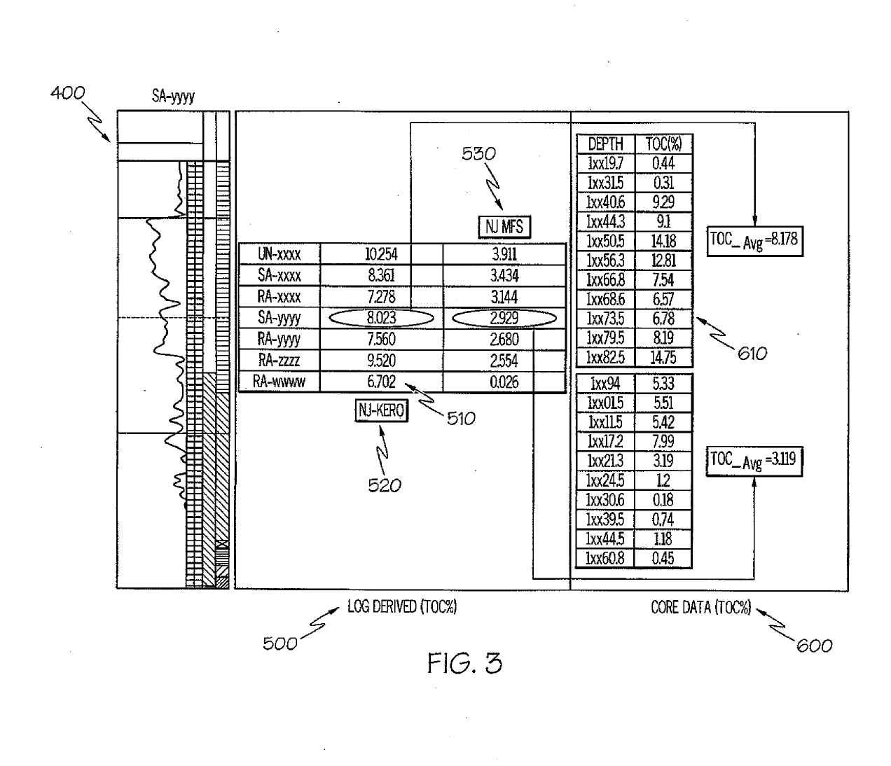

FIG. 5 illustrates transform function 70 for calculating the TOCDF from the system for determining the production success of wells 10 in FIG. 1. The density footage 60 may be input into transform function 70 to produce the total organic content per density footage 80, or TOCDF. The TOCDF calculation may determine the production success of wells with 100% accuracy. Transform function 70 may provide an accurate estimation of total organic carbon content of a reservoir unit.

When the TOCDF factor of a reservoir unit is over 2.45 weight %, the organic content of the unit may be too high and the unit may not produce liquid hydrocarbons. The reason for this may be the composition of bitumen in the high organic rich reservoir regions. Bitumen is a highly viscous unconventional petroleum deposit, and is not easily extracted with conventional liquid oil drilling processes. Reservoir regions containing high TOCDF values may be better suited for unconventional extraction techniques.

Conventional liquid hydrocarbons may be extracted from those regions with a TOCDF factor close to or equal to 2.45 weight %. The TOCDF factor may be useful for marking these areas with higher organic content for later extraction with other extraction techniques like hydraulic fracking, or other techniques that may render extraction economically viable.

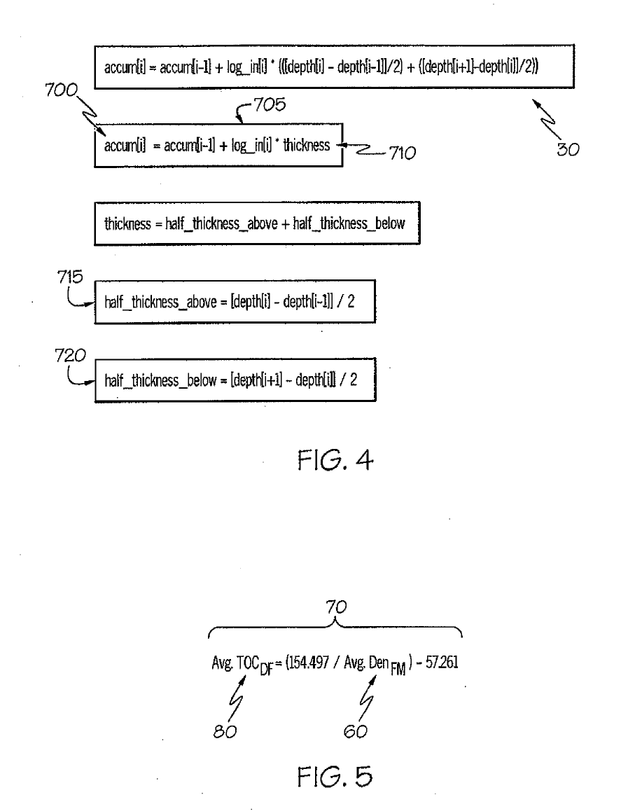

FIG. 6 illustrates a productivity index formula for calculating the expected productivity of a well. In certain configurations of the system for determining the production success of wells, the productivity index may provide a measure to distinguish wells that may be identified as capable of producing hydrocarbons. The productivity index may measure an exponential increase in the productivity of a well as the TOCDF increases linearly. Once the TOCDF is obtained from the transform equation, the result may be input into the productivity index formula in order to obtain an expected productivity for hydrocarbon output of the well. The productivity index may comprise the ratio of the total liquid flow rate to the pressure drawdown. The productivity index may be calculated as a part of the system for determining the production success of wells.

The productivity index may aid well planning and project scheduling. For example, if the productivity index indicates a high level of production, extraction resources may be reserved for longer, and more drilling resources may be allocated in that particular region. If the productivity index for a series of locations indicates low productivity indexes, resources may be moved or scheduled for extraction of more productive regions.

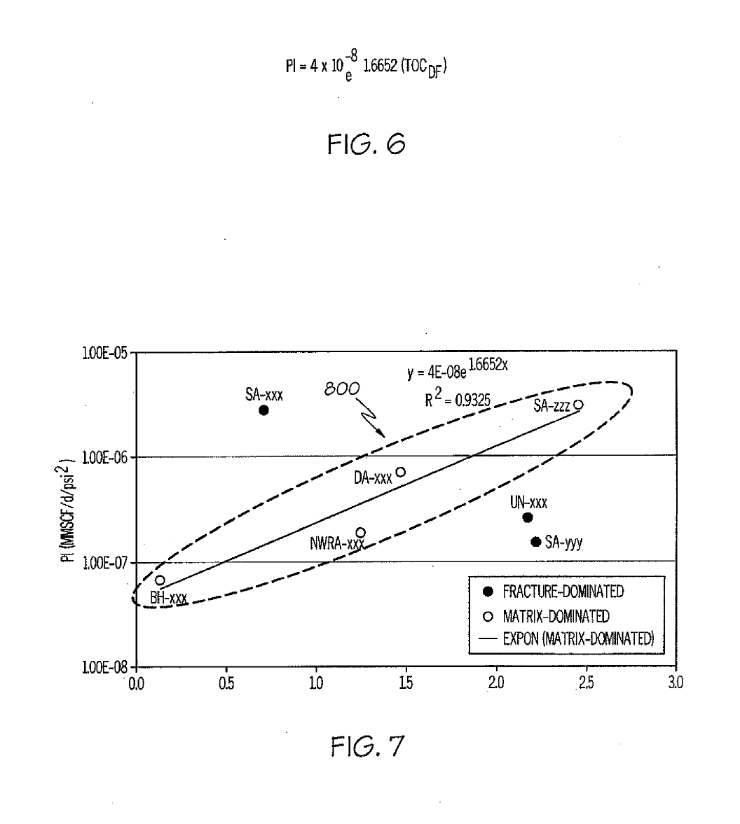

FIG. 7 illustrates a plot of the productivity index against TOCDF for successful wells. A generalized trend line 800 may indicate that as TOCDF levels increase up to 2.45%, the productivity index of the well increases. Using this information, well sites may be planned in regions with TOCDF levels equal to or below 2.45 weight %. More productivity may be expected in regions with TOCDF levels at or below 2.45 weight %. The measurements in FIG. 7 were calculated at each successful well site. Trend line 800 may suggest a productivity estimation for future well sites in accordance with the productivity index from FIG. 6.

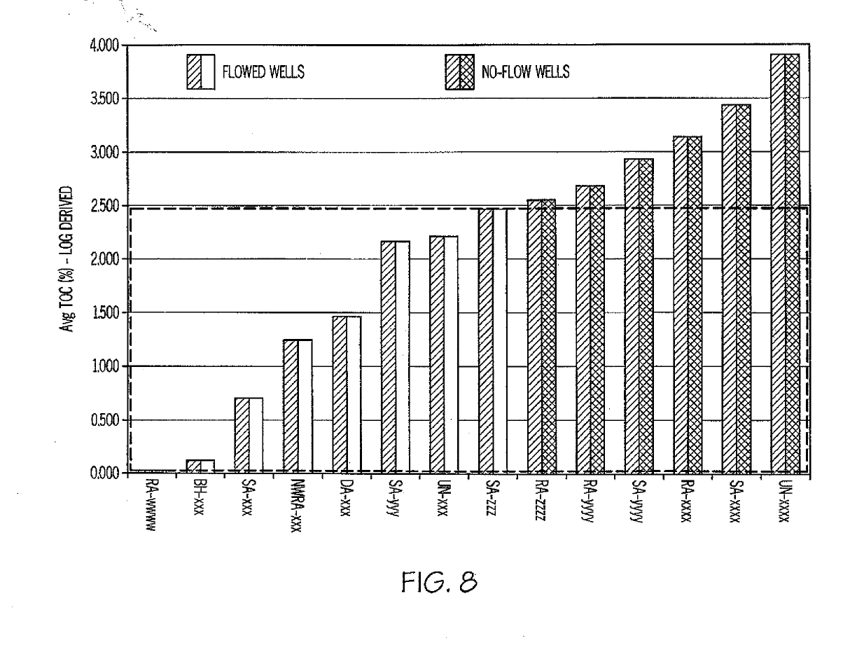

FIG. 8 illustrates a histogram plot of average TOCDF of the resource plays from system from determining the production success of wells 100 from FIG. 1. The histogram may indicate that TOCDF may influence whether a well will produce hydrocarbons. The cut off for production may occur in wells where the lower organic rich laminated unit may contain 2.45% TOCDF. The TOCDF histogram may provide a reliable production success indicator. FIG. 8 may show the production success cut off at 2.45 weight %.

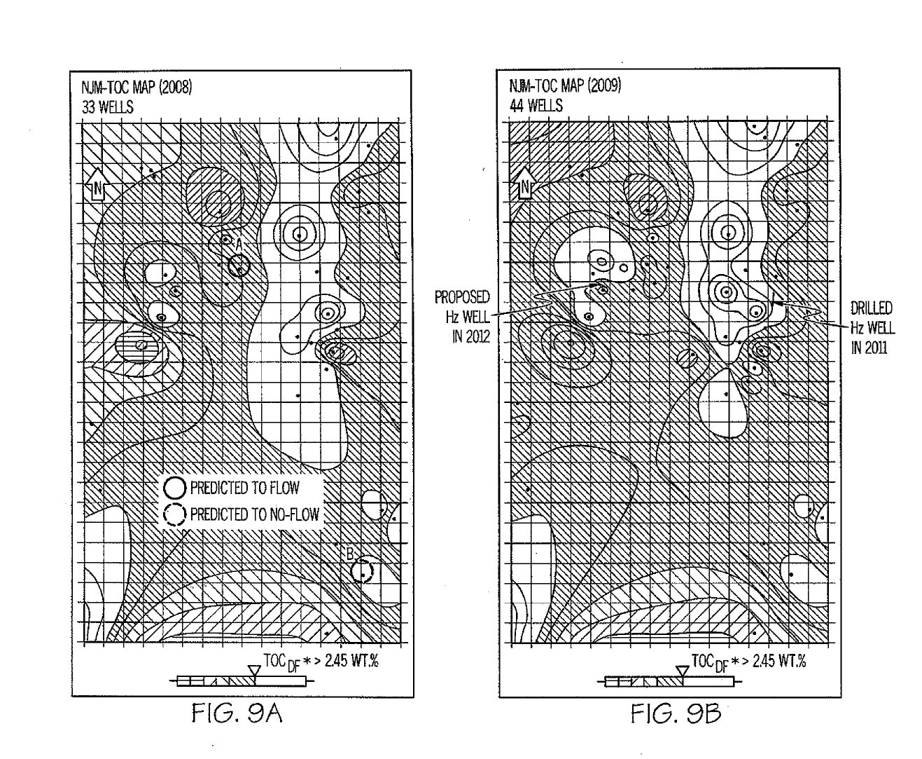

FIGS. 9A-B illustrate a TOCDF value contour map indicating the TOCDF levels of a reservoir unit across a sample region. FIG. 9A illustrates predicted well sites, while FIG. 9B illustrates drilled well sites in accordance with a particular embodiment of the disclosure. The reservoir unit may comprise a lower organic rich laminated layer. The dark areas may indicate regions where TOCDF values are low. These areas may indicate hydrocarbon production zones, but may not indicate regions where strong productivity is expected. The lighter gray areas may indicate regions where the TOCDF level approaches 2.45 weight %. These areas may contain the highest productive TOCDF content, and may be expected to produce effectively. The white areas indicate regions where production is not expected. These may comprise regions containing TOCDF content above 2.45 weight %. The present embodiment may allow managers and engineers to plan well placement strategies based on information contained in the map, and effectively exploit hydrocarbon resources across previously unpredictable hydrocarbon reservoirs.

The map in FIG. 9A may further be useful for determining which areas will support hydrocarbon extraction efforts. If reservoir indications are not promising, as indicated by an area that is too dark or too light, production management may relocate drilling resources to a more sustainable region.

For example, the TOCDF map in FIG. 9A for the unit based on well data control (although very sparse) may be prepared to plot the prospective area of predicted flow and no-flow (white area with TOCDF >2.45 weight %) rates of wells and their distribution trend (FIG. 7). These maps may be used for focused horizontal well placement and release for targeting reservoirs along with other well location release criteria. The thickness of these formation units may be very low (30 ft.) and may be below the seismic resolution, particularly when seismic image may be limited by greater depth of occurrence and sub-salt imaging with varied salt thicknesses (200 ft.-850 ft.) in the study area.

The study in the above example may have established an empirical relation between TOCDF and the initial productivity index of the well calculated from the successfully flowed and tested wells, especially for predicting initial productivity indexes from log derived TOCDF in the matrix support dominated dual porosity flow systems in kerogen rich, unconventional, and fractured limestone reservoir complexes. Similar studies may be carried out using the suggested workflow and may define the reservoir parameter TOCDF. The studies may further establish the relationship of the controlling factor with DST results. Thus, application of the new approach may be extended to other reservoirs that may have been reported with similar DST flow and no-flow trends.

While the disclosure has been described in connection with various embodiments, it will be understood by those of ordinary skill in the art that other variations and modifications of the various embodiments described above may be made without departing from the scope of the disclosure. Other embodiments will be apparent to those of ordinary skill in the art from a consideration of the specification or practice of the embodiments of the disclosure disclosed herein. The specification and the described examples are considered as exemplary only, with the true scope and spirit of the embodiments of the disclosure indicated by the following claims.

What is claimed is:

1. A system for determining hydrocarbon production, comprising:
one or more processors; and
a memory comprising logic operable to:
determine an accumulated unit density of a reservoir unit from a density log;
determine a thickness of the reservoir unit;
determine a density footage of the reservoir unit based on the accumulated density and the thickness;
generate a total organic carbon content per density footage based on the density footage; and
determine a potential production of hydrocarbons based on the total organic carbon content per density footage of the reservoir unit.

2. The system of claim 1, wherein the potential production of hydrocarbons is determined if the total organic content per density footage is less than 2.45 weight %.

3. The system of claim 1, wherein the memory comprising logic is further operable to calculate a hydrocarbon productivity score.

4. The system of claim 1, wherein the total organic carbon content per density footage is generated by applying the formula
TOCDF=(154.497/Avg. Den_{FM})−57.261
and wherein
TOCDF=the total organic carbon content per density footage; and
Avg. Den_{FM}=the density footage.

5. The system of claim 1, wherein the density footage is determined by dividing the accumulated density by the thickness.

6. The system of claim 1, wherein the density log is generated by a device comprising a logging tool.

7. The system of claim 1, wherein the density log is generated by a device comprising a logging while drilling device.

8. A method for determining hydrocarbon production comprising:
determining an accumulated density of a reservoir unit from a density log;
determining a thickness of the reservoir unit;
determining a density footage of the reservoir unit based on the accumulated density and the thickness;
generating a total organic carbon content per density footage of the reservoir unit based on the density footage; and
determining a potential production of hydrocarbons based on the total organic carbon content per density footage of the reservoir unit.

9. The method of claim 8, wherein the potential production of hydrocarbons is determined if the total organic content per density footage is less than 2.45 weight %.

10. The method of claim 8, wherein the reservoir unit comprises a lower organic rich laminated reservoir unit.

11. The method of claim 8, wherein generating the total organic carbon content per density footage comprises applying the formula
TOCDF=(154.497/Avg. Den_{FM})−57.261
and wherein
TOCDF=the total organic carbon content per density footage; and
Avg. Den_{FM}=the density footage.

12. The method of claim 8, wherein the density footage is determined by dividing the accumulated density by the thickness.

13. The method of claim 8, wherein the density log is generated by a device comprising a logging tool.

14. The method of claim 8, wherein the density log is generated by a device comprising a logging while drilling device.

15. A method, comprising:
determining an accumulated density of a reservoir unit from a density log;
determining a thickness of the reservoir unit;
determining a density footage of the reservoir unit based on the accumulated density and the thickness;
generating a total organic carbon content per density footage of the reservoir unit based on the density footage;
generating a hydrocarbon productivity score based on the total organic carbon content per density footage; and
transmitting the hydrocarbon productivity score in a displayable format.

16. The method of claim 15, wherein generating the total organic carbon content per density footage comprises applying the formula
TOCDF=(154.497/Avg. Den_{FM})−57.261
and wherein
TOCDF=the total organic carbon content per density footage; and
Avg. Den_{FM}=the density footage.

17. The method of claim 15, wherein the density footage is determined by dividing the accumulated density by the thickness.

18. The method of claim 15, wherein generating the hydrocarbon productivity score comprises applying the formula
PI=4×10⁻⁸e^{1.6652(TOCDF)}
and wherein
TOCDF=the total organic carbon content per density footage; and
PI=the hydrocarbon productivity score.

19. The method of claim 15, wherein the density log is generated by a device comprising a logging tool.

20. The method of claim 15, wherein the density log is generated by a device comprising a logging while drilling device.
